(19) United States
(12) Patent Application Publication
(10) Pub. No.: US 2018/0339149 A1
(43) Pub. Date: Nov. 29, 2018

(54) MULTI-CONTACT ELECTRODE

(71) Applicant: Heraeus Deutschland GmbH & Co. KG, Hanau (DE)

(72) Inventors: Robert DITTMER, Hanau (DE); Ulrich HAUSCH, Frankfurt am Main (DE); Jens TRÖTZSCHEL, Ronneburg (DE)

(73) Assignee: Heraeus Deutschland GmbH & Co. KG, Hanau (DE)

(21) Appl. No.: 15/986,991

(22) Filed: May 23, 2018

(30) Foreign Application Priority Data
May 24, 2017 (EP) 17172631.8

(51) Int. Cl. A61N 1/05; A61N 1/36

(52) U.S. Cl. CPC A61N 1/0534; H05K 1/0306; A61N 1/3605

(57) ABSTRACT

One aspect relates to a multi-contact electrode, a method for manufacturing a multi-contact electrode, and a use of such multi-directional multi-contact electrode. The multi-contact electrode includes a support structure and a plurality of electrically conductive electrode segments. The support structure is made of a ceramic material. The electrode segments are made of a cermet material and are supported by the support structure. The electrode segments are distributed over an outer surface of the multi-contact electrode to form a multi-directional multi-contact electrode.

### CROSS-REFERENCE TO RELATED APPLICATION

This Utility patent application claims priority to Application No. EP 17172631.8, filed on May 24, 2017, which is incorporated herein by reference.

### BACKGROUND

One aspect relates to a multi-contact electrode, a method for manufacturing a multi-contact electrode, and a use of such multi-directional multi-contact electrode.

So-called multi-contact electrodes are used primarily in the field of neurostimulation especially in brain and deep brain stimulation and used for directional pacing. This kind of lead-tip electrode features two or more pairs of ring-electrode halves that are connected with a wire, coil or strand. The electrodes are typically micro-machined and the leads are laser welded. In order to insulate the electrodes from each other, they are overmolded with a non-conductive polymer.

US 2015/000124 A1 discloses a method of manufacturing a device for brain stimulation which includes forming a polymeric lead body having a distal end section and coupling at least one metallic pre-electrode to the distal end section of the lead body. The pre-electrode defines a divider with a plurality of partitioning arms and has a plurality of fixing lumens. A portion of the pre-electrode aligned with the portioning arms is removed to divide the pre-electrode into a plurality of segmented electrodes. Each of the plurality of segmented electrodes defines at least one of the plurality of fixing lumens at least partially disposed through the segmented electrode. A material is introduced through the at least one fixing lumen to couple the plurality of segmented electrodes to the lead body.

Such method of fabrication can have numerous disadvantages, as, for example, a complex fabrication procedure, a limited miniaturization potential, a limited number of segmented electrode rings and, therefore, a limited resolution for the therapy. Hence, there may be a need to provide a multi-contact electrode which can be easily manufactured. For these and other reasons, a need exists for the present embodiments.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIGS. 1a and 1b illustrate schematically and exemplarily an embodiment of a multi-contact electrode according to one embodiment,

FIGS. 2a and 2b illustrate schematically and exemplarily another embodiment of a multi-contact electrode according to one embodiment,

FIGS. 3a and 3b illustrate schematically and exemplarily another embodiment of a multi-contact electrode according to one embodiment,

FIGS. 4a to 4d illustrate schematically and exemplarily a method to attach wires to a multi-contact electrode, and

FIG. 5 illustrates a schematic overview of steps of a method for manufacturing a multi-contact electrode according to one embodiment.

### DETAILED DESCRIPTION

In the following Detailed Description, reference is made to the accompanying drawings, which form a part hereof, and in which is illustrated by way of illustration specific embodiments in which one embodiments may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present embodiments. The following detailed description, therefore, is not to be taken in a limiting sense, and the scope of the present embodiments are defined by the appended claims.

It is to be understood that the features of the various exemplary embodiments described herein may be combined with each other, unless specifically noted otherwise.

Some of the above-problems are solved by the subject-matters of the independent claims, wherein further embodiments are incorporated in the dependent claims. It should be noted that the aspects described in the following apply also to the multi-contact electrode, the method for manufacturing a multi-contact electrode, and the use of such multi-directional multi-contact electrode.

According to one embodiment, a multi-contact electrode is presented. The multi-contact electrode includes a support structure and a plurality of electrically conductive electrode segments. The support structure is made of a ceramic material. The electrode segments are made of a cermet material and are supported by the support structure. The electrode segments are distributed over an outer surface of the multi-contact electrode to form a multi-directional multi-contact electrode.

The electrode segments may form very fine leads or channels within the multi-contact electrode. Thereby, the multi-contact electrode allows an internal routing, which means a transfer of signals within the electrode body. Further, the fine leads or channels may form a unitary part with the multi-contact electrode and therefore no assembly of components is necessary.

The ceramic material may include or be alumina for example, alumina with high purity, in one embodiment of at least 96 wt.-% Al, and in one embodiment at least 99.6 wt.-% Al. The cermet material may be a mixture of a ceramic and a metal, for example, alumina and platinum. Other materials will be described in detail further below

As plurality, the multi-contact electrode may include at least two, in one embodiment at least four, and in one embodiment at least eight electrically conductive electrode segments.

For a cylindrical multi-contact electrode, the outer surface is the circumference of the multi-contact electrode. The wording that the electrode segments are distributed over the outer surface of the multi-contact electrode can be understood in that the electrode segments are physically spaced apart from each other and/or electrically isolated from each other. The distribution allows forming the multi-directional multi-contact electrode, which can be understood as an electrode with multiple electric contacts, which are directed in several, different directions.

As will be described in detail further below, the multi-directional multi-contact electrode may be manufactured by a High Temperature Cofired Ceramics (HTCC) method in which the device is made by processing a number of layers independently and assembling them into the device as a final step. In an example, the multi-contact electrode then includes a stack of layers each comprising a portion of a support structure and/or a portion of at least one of the electrode segments. The stack of layers may include at least two, in one embodiment at least five, and in one embodiment at least ten single layers. In an example, the stack of layers includes between 2 and 14 layers per millimeter and in one embodiment between 4 and 10 layers per millimeter.

The multi-directional multi-contact electrode may also be manufactured by an additive manufacturing method in which the support structure and the electrode segments or at least portions thereof are formed together and at the same time.

In an example, the multi-contact electrode is then a monolithic structure, which means a one-piece structure not comprising several layers or components. As a result, no assembly of components is necessary.

The structure and the materials of the multi-directional multi-contact electrode according to one embodiment allow using alternative, very effective and very flexible manufacturing methods. The costs of the multi-directional multi-contact electrode may thereby be reduced. Further, multi-directional multi-contact electrodes with various designs, structures and dimensions can be easily provided. The multi-directional multi-contact electrodes can be miniaturized. Instead or additionally, a density of functional structures as, for example, a number of single electrodes, electrode segments, electric paths etc. can be increased. Further, the multi-directional multi-contact electrode and for example, the support structure can be made harder and therefore more stable and robust. Furthermore, surface properties as, for example, a good micro roughness can be easily achieved.

The multi-directional multi-contact electrode provides electrical contacts at the outer surface or circumference of the electrode in order to allow for a transmission of signals, for example, for sensing or stimulating. In order to increase an effectiveness of therapy, that is, minimizing a rate of non-responders and eliminating unwanted side effects, the targetability is improved by increasing a number and density of individual contacts to, for example, 16 or higher (in contrast to conventionally 8) without enlarging a geometrical footprint of the part. At the same time, the manufacturing is simplified and the conventionally used polymeric insulator is replaced by a much more durable and highly resistant and mechanically more stable ceramic.

The multi-directional multi-contact electrode may have a circular, oval, square, rectangular, polygonal or the like cross section. The “corners” may be rounded or sharp.

The electrode segments are at the outer surface of the multi-contact electrode and may extend into the bulk of the multi-contact electrode, which means at least partially or completely in the direction of a center of the multi-contact electrode when seen in a cross section. For a, in a cross section, cylindrical multi-contact electrode, the electrode segments may be sectors or segments of a circle. For a, in a cross section, square, rectangular or polygonal multi-contact electrode, the electrode segments may form triangles, wedges, squares, rectangles, trapezoids or polygons.

In an example, the multi-contact electrode segments and for example, a surface of the multi-contact electrode segments is coated to provide a predetermined physical property of the multi-directional multi-contact electrode. The predetermined physical property may be a predetermined electrical resistance, surface roughness, friction coefficient and/or the like. In an example, an iridium oxide, iridium, platinum or TiN coating is applied to obtain a low contact impedance, for example, lower than 1500 Ohms/mm2. However, the outer surface of the multi-contact electrode may also be uncoated and provides such predetermined physical property without any coating.

In an example, the outer surface of the multi-contact electrode has a roughness Ra of at least 1 μm, in one embodiment of at least 0.5 μm.

In an example, an outer surface of the electrode segments has a porosity of 3% or less, in one embodiment 2% or less.

In an example, the multi-layer body has a main body with an elongated shape with opposing ends that include end surfaces. The main body may have three cross-sections that are mutually orthogonal to each other, where one cross-section has a periphery with at least four edges, each of the edges being oriented essentially perpendicular to two of the other edges, and the periphery of the other two cross-sections includes ellipses or sections of ellipses. The sections may be formed by means of one or two straight cutting lines, which in case of two cutting lines are parallel. Additionally to the main body, the multi-layer body may include a contact body at one end of the multi-layer body for contacting to, for example, a lead, and/or a front body at the other end of the multi-layer body with, for example, a rounded or tip-shaped front.

In an example, the multi-contact electrode includes at least one contacting portion with a deviating width. This deviating width deviates from a width of the bulk or main body of the multi-contact electrode. This means, the contacting portion may have a smaller or a larger width than the bulk of the multi-contact electrode. For example, the contacting portion is thinner than the main part of the multi-contact electrode and thereby forms a finger shaped contacting portion. In general, the contacting portion serves for contacting the multi-contact electrode. For example, wires can be attached to the finger shaped contacting portion by, for example, welding the wires along the length of the finger shaped contacting portion. Of course, the contacting portion can also have the same width as the bulk or main body of the multi-contact electrode. Then, the wires may be attached to and/or inserted into a front face of the contacting portion.

According to one embodiment, also a method for manufacturing a multi-contact electrode is presented. It includes the following steps, not necessarily in this order:

a) forming a support structure, and
b) forming a plurality of electrically conductive electrode segments.

The support structure is made of a ceramic material and the electrode segments are made of a cermet material. The support structure is arranged to support the electrode segments and the electrode segments are distributed over an outer surface of the multi-contact electrode to form a multi-directional multi-contact electrode.

The electrode segments may form very fine leads or channels within the multi-contact electrode to allow an internal routing. The fine leads or channels form a unitary part with the multi-contact electrode and therefore no further assembly of components is necessary. For example, no welding and overmolding is necessary.

In an example, the method for manufacturing a multi-contact electrode is a High Temperature Cofired Ceramics (HTCC) method in which the device is made by processing a number of layers independently and assembling them into the device as a final step. In an example, the forming of the support structure and/or the forming of the electrode segments therefore includes a forming of a layer, which is repeated to form a stack of layers each comprising a portion of a support structure and/or a portion of at least one of the electrode segments. In other words, a green ceramic matrix may be provided in which a cermet paste may be inserted and then a stack of several of these layers may be built up. The stack can then be sintered to firmly bond ceramic particles of the cermet with each other and with the ceramic particles of the ceramic matrix.

In an example, the stack of layers is formed along a longitudinal direction of the multi-contact electrode. This means, the multi-contact electrode is formed in an upright or vertical orientation. In another example, the stack of layers is formed along a direction perpendicular to the longitudinal direction of the multi-contact electrode. This means, the multi-contact electrode is formed in a lying or horizontal orientation.

The stack of layers may include at least two, in one embodiment at least five, and in one embodiment at least ten single layers. In an example, the stack of layers includes between 2 and 14 layers per millimeter and in one embodiment between 4 and 10 layers per millimeter.

In an example, the method for manufacturing a multi-contact electrode is an additive manufacturing method. In an example, the forming of the support structure and/or the forming of the electrode segments includes one of a group of printing, 3D printing, ceramic injection molding, co-extrusion, powder pressing and/or the like. The support structure and the electrode segments may then be simultaneously formed. This means the support structure and the electrode segments or at least portions thereof are formed together and at the same time in contrast to separate and independent. In other words, the support structure and the electrode segments directly form a 3D structure instead of a quasi 2D layer to be stacked into a 3D structure.

In an example, the method for manufacturing a multi-contact electrode further includes the step of isostatic pressing the support structure and the electrode segments.

In an example, the method for manufacturing a multi-contact electrode further includes the step of sintering the support structure and the electrode segments and for example, co-sintering or co-firing the support structure and the electrode segments.

The sintering step may be configured for a material bonding of metal particles and ceramic particles within the cermet material. The sintering step may also be configured for a material bonding of the ceramic material and the cermet material.

In an example, the method for manufacturing a multi-contact electrode further includes the step of inserting a pre-formed element in the support structure and/or the electrode segments for additive manufacturing methods as ceramic injection molding, co-extrusion, powder pressing and/or the like. The pre-formed element may for example be a single electrode or electrode segment.

In an example, the method for manufacturing a multi-contact electrode further includes the step of generating a final shape and/or dimension of the multi-contact electrode by means of mechanical and/or thermal processing.

In an example, the method for manufacturing a multi-contact electrode further includes the step of attaching a lead structure to the multi-directional multi-contact electrode, which may mean a method to attach several wires to the electrode. This attaching step includes a providing of a flat lead frame, which may be laser cut from a metal sheet. The attaching step further includes a bending of the lead frame into a round lead structure. The lateral ends of the lead frame may be joined together by, for example, laser welding. The attaching step further includes a fixing of a foot portion of the lead structure to the multi-directional multi-contact electrode. The foot portion may be laser welded to the electrode. The attaching step further includes a removing of a head portion of the lead structure by, for example, laser cutting. The attaching step may further include an overmoulding of the remaining lead structure and at least a portion of the multi-directional multi-contact electrode by a polymer, for example, a biocompatible polymer, to form a strain-relief for the leads. As a result, leads or wires are fixed and secured to the multi-directional multi-contact electrode.

According to one embodiment, also a multi-directional multi-contact electrode is presented which is manufactured as described above.

According to one embodiment, also a use of the multi-directional multi-contact electrode as described above and/or manufactured as described above as a pacing electrode for neurostimulation, brain and deep brain stimulation and/or the like is presented.

It shall be understood that the multi-contact electrode, the method for manufacturing a multi-contact electrode, and the use of such multi-directional multi-contact electrode according to the independent claims have similar and/or identical in one embodiment, for example, as defined in the dependent claims. It shall be understood further that one embodiment can also be any combination of the dependent claims with the respective independent claim.

These and other aspects will become apparent from and be elucidated with reference to the embodiments described hereinafter.

### Definitions

### Ceramic

A ceramic according to one embodiment can be any ceramic the skilled person deems applicable to one embodiment. In one embodiment ceramic is electrically insulating. The ceramic is in one embodiment selected from the group consisting of an oxide ceramic, a silicate ceramic and a non-oxide ceramic or a combination of at least two thereof. The oxide ceramic includes in one embodiment a metal oxide or a metalloid oxide or both. A metal of the metal oxide is in one embodiment selected from the group consisting of aluminum, zirconium, titanium, or a combination of at least two thereof. In one embodiment metal oxide is selected from the group consisting of aluminum oxide (Al2O3); magnesium oxide (MgO); zirconium oxide (ZrO2); yttrium oxide (Y2O3); aluminum titanate (Al2TiO5); silicon oxide (SiO2); a piezo ceramic as for example lead-zirconate (PbZrO3), lead-titanate (PbTiO3) and lead-zirconate-titanate (PZT); or a combination of at least two thereof. In one embodiment metalloid of the metalloid oxide is selected from the group consisting of boron, silicon, tellurium, or a combination of at least two thereof. One embodiment of oxide ceramic includes one selected from the group consisting of aluminum oxide toughened with zirconium oxide enhanced (ZTA—Zirconia Toughened Aluminum—Al2O3/ZrO2), zirconium oxide toughened with yttrium (Y-TZP), barium(Zr, Ti)oxide, barium(Ce, Ti)oxide or a combination of at least two thereof.

The silicate ceramic is in one embodiment selected from the group consisting of a steatite (Mg3[Si4O10(OH)2]), a cordierite (Mg, Fe2+)2(Al2Si)[Al2Si4O18]), a mullite (Al2Al2+2xSi2-2xO10-x with x=oxide defects per unit cell), a feldspar (Ba,Ca,Na,K,NH4)(Al,B,Si)4O8) or a combination of at least two thereof. The non-oxide ceramic in one embodiment includes a carbide or a nitride or both. In one embodiment carbide is one selected from the group consisting of silicon carbide (SiC), boron carbide (B4C), titanium carbide (TiC), tungsten carbide, cementite (Fe3C) or a combination of at least two thereof. In one embodiment nitride is one selected from the group consisting of silicon nitride (Si3N4), aluminum nitride (AlN), titanium nitride (TiN), silicon aluminum oxinitride (SIALON) or a combination of at least two thereof. A further embodiment non-oxide ceramic is sodium-potassium niobate.

Further, the ceramic according to one embodiment may be or include glass or glass ceramic.

### Cermet

According to one embodiment, a cermet is a composite material comprising at least one metallic component in a least one ceramic matrix. At least one ceramic powder and at least one metallic powder can for example be applied for preparing a cermet, wherein to at least one of the powders for example a binder can be added and optionally at least one surfactant. The ceramic powder/the ceramic powders of the cermet in one embodiment have a median grain size of less than 10 μm, in one embodiment less than 5 μm, in one embodiment less than 3 μm. In some cases the ceramic powder of the cermet has an average particle size of at least 15 μm. The metallic powder/the metallic powders of the cermet in one embodiment have an average grain size of less than 15 μm, in one embodiment less than 10 μm, in one embodiment less than 5 μm. Therein, the average grain size is particularly the median value or the D50. The D50 gives the value, at which 50% of the grains of the ceramic powder and/or the metallic powder are smaller than the D50. In one embodiment cermet is characterized by a high specific conductivity, which is in one embodiment at least 1 S/m, in one embodiment at least 103 S/m, in one embodiment at least 104 S/m. The at least one ceramic component of the cermet according to one embodiment includes a ceramic according to one embodiment. The at least one metallic component of the cermet according to one embodiment includes in one embodiment one selected from the group consisting of platinum, iridium, niobium, palladium, iron, stainless steel, a cobalt-chromium-alloy, molybdenum, tantalum, tungsten, titanium, cobalt and zirconium and gold or a combination of at least two thereof. Therein in one embodiment combination is an alloy. In one embodiment stainless steel is stainless steel 316L. Generally, the cermet becomes electrically conductive if the metal content of the cermet is above the so called percolation threshold, at which metal particles in the sintered cermet are at least partly connected to each other in such a way that electrical charges can be transported via conduction. Therefore, the metal content of the cermet should, depending on the choice of materials, be at least 25 vol.-%, in one embodiment at least 32 vol.-%, in one embodiment at least 38 vol.-%, each based on the total volume of the cermet.

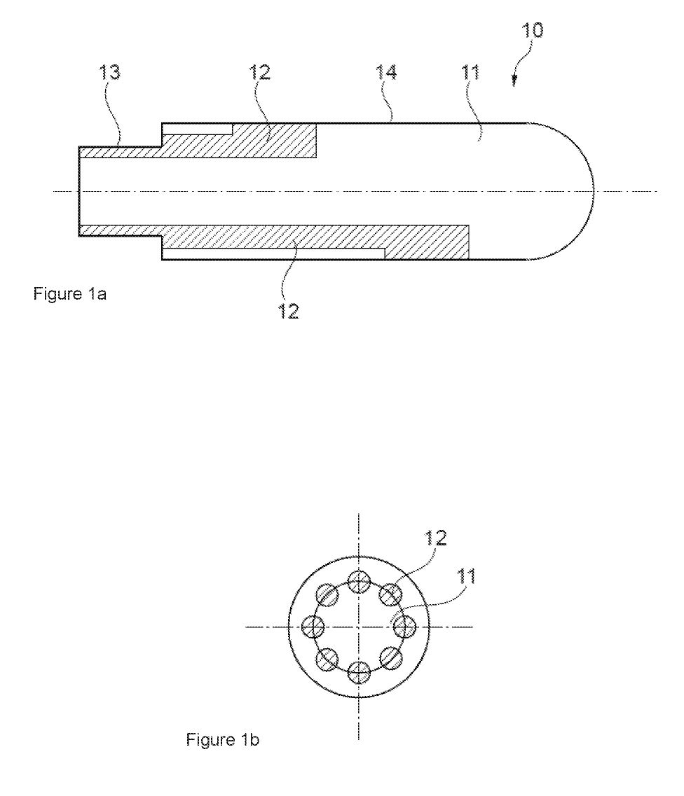

FIGS. 1a and 1b illustrate schematically and exemplarily an embodiment of a multi-contact electrode 10 according to one embodiment in a cross sectional view and in a front view. The multi-contact electrode 10 includes a support structure 11 and a plurality of electrically conductive electrode segments 12. The support structure 11 is made of a ceramic material. The electrode segments 12 are made of a cermet material and are supported by the support structure 11. The electrode segments 12 are distributed over an outer surface of the multi-contact electrode 10 to form a multi-directional multi-contact electrode 10.

At its free or distal end, eight individually accessible electrode segments 12 are illustrated. The body or proximal part of the multi-contact electrode 10 illustrates two rings of electrode segments 12. Each ring includes four electrode segments 12. The electrode segments 12 of different rings are displaced relative to each other. Of course, they can also be arranged in an aligned manner.

The multi-contact electrode 10 may be configured for use within a human body. Within a human body, each of the electrode segments 12 may be used for directional stimulation or positional feedback sensing. In contrast to a single ring electrode that spans an entire 360° circumference, the present multi-contact electrode 10 includes electrode segments 12 which only span a portion of the circumference (for example, 180°, 90° degrees or less), such that directional stimulation or positional feedback sensing can be much more precisely controlled relative to a given target within the human body.

The multi-contact electrode 10 allows its manufacture with an increased density of electrode segments 12 in contrast to conventional electrodes. Increased density of electrode segments 12 is useful in a variety of applications. For example, the multi-contact electrode 10 can be used in deep brain stimulation, in which the multi-contact electrode 10 delivers electrical pulses into one or several specific sites within the brain of a patient to treat various neurological disorders, such as chronic pain, tremors, Parkinson's disease, dystonia, epilepsy, depression, obsessive-compulsive disorder, and other disorders. In another applications, the multi-contact electrode 10 may be configured for spinal cord stimulation, peripheral nerve stimulation, dorsal root stimulation, cortical stimulation, ablation therapies, cardiac rhythm management leads, various catheter configurations for sensing, and various other therapies where directional sensing or stimulation are needed.

The increased package density of electrical contacts within the multi-contact electrode 10 may be achieved by a combination of a ceramic support structure 11 and cermet electrode segments 12. For example by means of a printing and co-firing process, platinum-containing electrically conductive pathways and contacts can be integrated into an insulating ceramic support structure 11. By using fine-scaled pathways and contacts as well as intricate machining, small three-dimensional parts may be manufactured in a cost effective and still very flexible manner.

The multi-contact electrode 10 according to one embodiment may provide at least one of the following advantages:
High potential for miniaturization, that is, the current size can be diminished which may have benefits for the patient (for example, improved comfort) and allows an implantation surgery with less invasive impact.
Increased number of contacts at the same size for increased therapy efficiency.
High degree of geometrical freedom: the shape of the electrical contacts can be largely varied and one is not bound to, for example, rectangular shapes.
Decreased single part count yields a more cost-efficient assembly and increased safety due to the decreased number of potential failure modes due to reduced single part count.
Mechanically highly robust when compared to a conventional polymer-based solution.

In FIG. 1, the multi-contact electrode 10 includes a contacting portion 13 with a deviating width, which means the contacting portion 13 has a smaller diameter (for example, 0.6 mm) than the main part 14 (for example, 1.2 mm) of the multi-contact electrode 10. In other words, the contacting portion 13 is thinner than the main part 14 of the multi-contact electrode 10 and thereby forms a finger shaped contacting portion 13. The finger may have a length of for example, 0.7 mm, while the main part 14 may have a length of for example, 3.5 mm. The contacting portion 13 serves for an easy contacting of the multi-contact electrode 10 in that wires (not illustrated) may be welded, glued, bonded or the like to the finger shaped contacting portion 13.

Of course, the contacting portion 13 can also have the same width as the main part 14 or bulk of the multi-contact electrode 10.

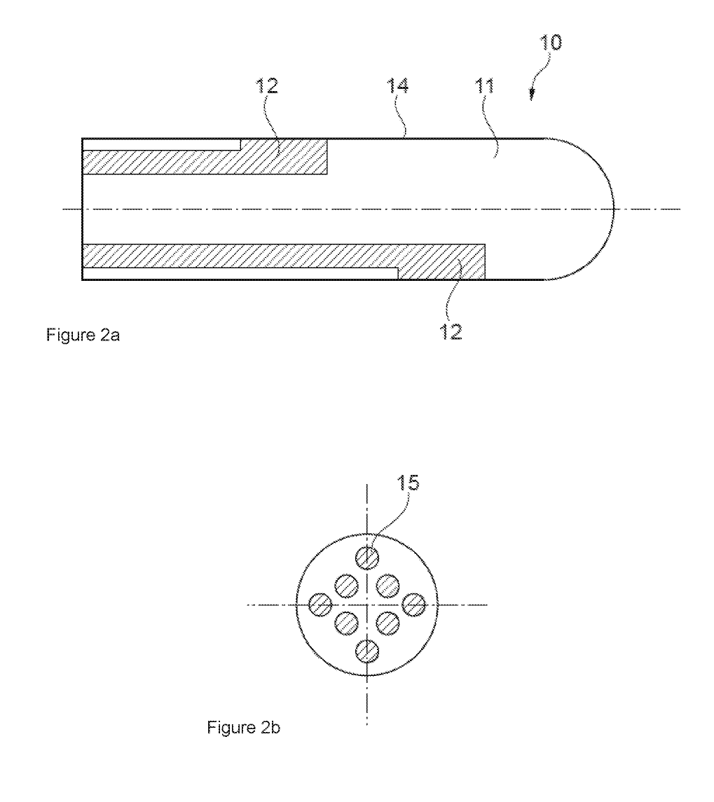

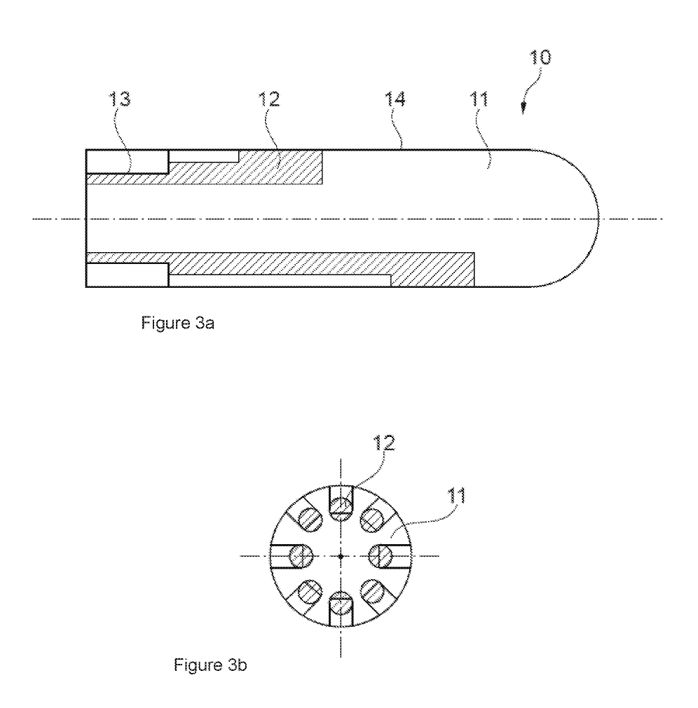

FIGS. 2a, 2b, 3a and 3b illustrate schematically and exemplarily other embodiments of a multi-contact electrode 10 according to one embodiment in a cross sectional view and in a front view. In FIGS. 2a and 2b, wires (not illustrated) may be inserted into holes 15 in a front face of the contacting portion. FIGS. 2a and 2b only illustrate the support structure 11 with recesses for the electrode segments 12.

In FIGS. 3a and 3b, wires (not illustrated) may be inserted into longitudinally open slits starting at the front face and extending at least partially along the contacting portion.

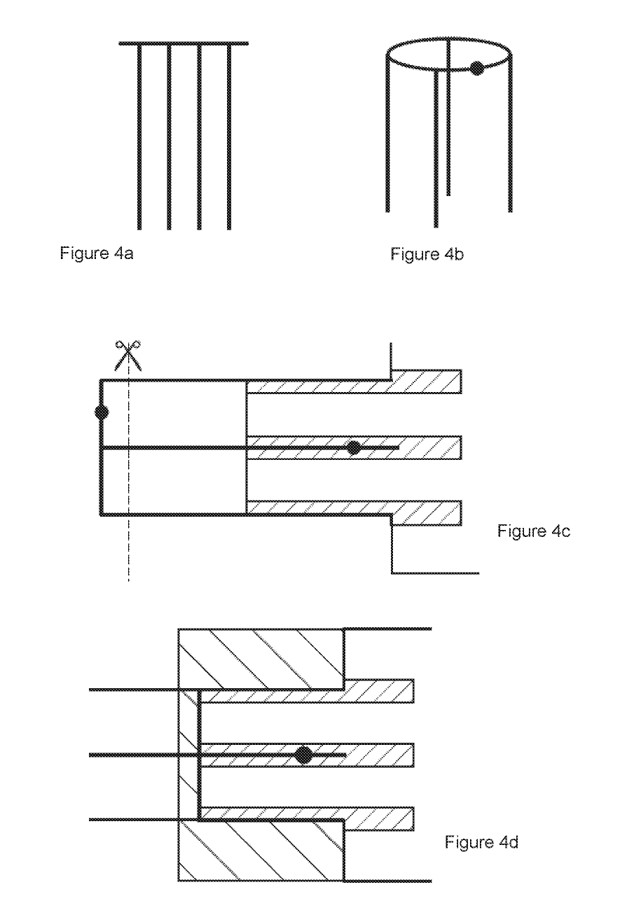

FIGS. 4a to 4d illustrate schematically and exemplarily a method to attach wires to a multi-contact electrode as, for example, illustrated in FIG. 3. In FIG. 4a, a leadframe is laser-cut from a metal sheet and in FIG. 4b, the leadframe is bended and joined by laserwelding. Now it is rather round than flat. In FIG. 4c, the leadframe is put over an electrode, fixed with a laserweld and a top portion of the leadframe is removed by, for example, lasercutting. In FIG. 4d, the lead-frame and the electrode are overmolded with, for example, a biocompatible polymer to form a strain-relief for the wire. The result are wires fixed and secured to the cermet-ceramic-composite electrode.

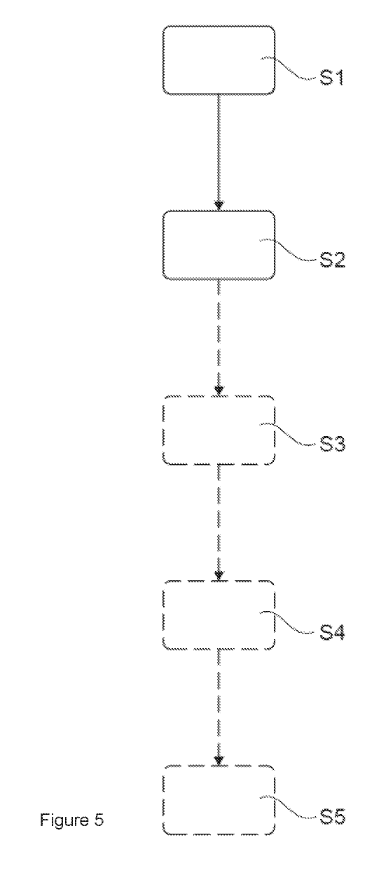

FIG. 5 illustrates a schematic overview of method steps for manufacturing a multi-contact electrode 10 according to one embodiment. The method includes the following steps, not necessarily in this order:

In a first step S1, forming a support structure 11.

In a second step S2, forming a plurality of electrically conductive electrode segments 12.

The support structure 11 is made of a ceramic material and the electrode segments 12 are made of a cermet material. The support structure 11 is arranged to support the electrode segments 12 and the electrode segments 12 are distributed over an outer surface of the multi-contact electrode 10 to form a multi-directional multi-contact electrode 10.

The method for manufacturing a multi-contact electrode 10 may be a High Temperature Cofired Ceramics (HTCC) method in which the device is made by processing a number of layers independently and assembling them into the device as a final step. In an example, the forming of the support structure 11 and/or the forming of the electrode segments 12 therefore includes a forming of a layer, which is repeated to form a stack of layers each comprising a portion of a support structure 11 and/or a portion of at least one of the electrode segments 12. The stack of layers may be either formed along a longitudinal direction of the multi-contact electrode 10 or along a direction perpendicular to the longitudinal direction of the multi-contact electrode 10.

The method for manufacturing a multi-contact electrode 10 may also be an additive manufacturing method as, for example, 3D printing, ceramic injection molding, co-extrusion, powder pressing and/or the like. The support structure 11 and the electrode segments 12 may then be formed together at the same time.

Method steps S1 and S2 may be followed by a step S3 of isostatic pressing of the support structure 11 and the electrode segments 12 and/or a step S4 of sintering the support structure 11 and the electrode segments 12 and/or a step S5 of generating a final shape and/or dimension of the multi-contact electrode 10 by means of mechanical and/or thermal processing.

It has to be noted that embodiments are described with reference to different subject matters. For example, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While one embodiment has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfil the functions of several items re-cited in the claims. The mere fact that certain measures are re-cited in mutually different dependent claims does not indicate that a combination of these measures cannot be used to advantage. Any reference signs in the claims should not be construed as limiting the scope.

Although specific embodiments have been illustrated and described herein, it will be appreciated by those of ordinary skill in the art that a variety of alternate and/or equivalent implementations may be substituted for the specific embodiments illustrated and described without departing from the scope of the present embodiments. This application is intended to cover any adaptations or variations of the specific embodiments discussed herein. Therefore, it is intended that these embodiments be limited only by the claims and the equivalents thereof.

What is claimed is:

1. A multi-contact electrode comprising:
a support structure, and
a plurality of electrically conductive electrode segments, wherein the support structure is made of a ceramic material, wherein the electrode segments are made of a cermet material, wherein the electrode segments are supported by the support structure, and wherein the electrode segments are distributed over an outer surface of the multi-contact electrode to form a multi-directional multi-contact electrode.

2. The multi-contact electrode of claim 1, wherein the multi-contact electrode comprises a stack of layers each comprising a portion of a support structure and/or a portion of at least one of the electrode segments.

3. The multi-contact electrode of claim 2, wherein the stack of layers comprises between 2 and 14 layers per millimeter.

4. The multi-contact electrode of claim 1, wherein the multi-contact electrode is a monolithic structure.

5. The multi-contact electrode of claim 1, wherein the outer surface of the multi-contact electrode has a roughness Ra of at least 1 μm.

6. The multi-contact electrode of claim 1, wherein an outer surface of the electrode segments has a porosity of 3% or less.

7. The multi-contact electrode of claim 1, wherein the ceramic material comprises alumina and the cermet material comprises alumina and platinum.

8. The multi-contact electrode of claim 1, wherein the multi-contact electrode is coated to provide a predetermined physical property of the multi-directional multi-contact electrode, a predetermined electrical resistance, surface roughness and/or friction coefficient.

9. The multi-contact electrode of claim 1, wherein the multi-layer body has a main body with an elongated shape with opposing ends that comprise end surfaces, the body having three cross-sections that are mutually orthogonal to each other, where one cross-section has a periphery with at least four edges, each of the edges being oriented essentially perpendicular to two of the other edges, and the periphery of the other two cross-sections comprises ellipses or sections of ellipses, where the sections are formed by means of one or two straight cutting lines, which in case of two cutting lines are parallel.

10. The multi-contact electrode of claim 1, comprising at least one contacting portion with a deviating width, which deviates from a bulk width of the multi-contact electrode.

11. The multi-contact electrode of claim 1 configured as a multi-directional multi-contact electrode as a pacing electrode for neurostimulation and/or deep brain stimulation.

12. A method for manufacturing a multi-contact electrode, comprising:
forming a support structure, and
forming a plurality of electrically conductive electrode segments, wherein the support structure is made of a ceramic material, wherein the electrode segments are made of a cermet material, wherein the support structure is arranged to support the electrode segments, and wherein the electrode segments are distributed over an outer surface of the multi-contact electrode to form a multi-directional multi-contact electrode.

13. The method of claim 12, wherein the forming of the support structure and/or the forming of the electrode segments comprises a forming of a layer, which is repeated to form a stack of layers each comprising a portion of a support structure and/or a portion of at least one of the electrode segments.

14. The method of claim 12, wherein the stack of layers is formed along a longitudinal direction of the multi-contact electrode.

15. The method of claim 13, wherein the stack of layers is formed along a direction perpendicular to the longitudinal direction of the multi-contact electrode.

16. The method of claim 12, wherein the forming of the support structure and/or the forming of the electrode segments comprises one of a group of printing, 3D printing, ceramic injection molding, co-extrusion, and powder pressing.

17. The method of claim 12 further comprising:
isostatic pressing the support structure and the electrode segments.

18. The method of claim 12 further comprising:
sintering the support structure and the electrode segments.

19. The method of claim 18, wherein the sintering step is configured for a material bonding of metal particles and ceramic particles within the cermet material.

20. The method of claim 18, wherein the sintering step is configured for a material bonding of the ceramic material and the cermet material.

21. The method of claim 12 further comprising:
attaching a lead structure to the multi-directional multi-contact electrode, wherein the attaching step comprises a providing of a flat lead frame, a bending of the lead frame into a round lead structure, a fixing of a foot portion of the lead structure to the multi-directional multi-contact electrode, and a removing of a head portion of the lead structure.
